(19) United States
(12) Patent Application Publication
(10) Pub. No.: US 2003/0181341 A1
(43) Pub. Date: Sep. 25, 2003

(54) Liquid personal cleansing compositions containing acyl sarcosinates

(75) Inventor: Naohisa Yoshimi, Nishinomiya (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/391,831

(22) Filed: Mar. 19, 2003

Related U.S. Application Data
(60) Provisional application No. 60/365,455, filed on Mar. 19, 2002.

(51) Int. Cl. A61K 7/50

(52) U.S. Cl. 510/156; 510/424; 510/426; 510/428

(57) ABSTRACT

A liquid personal cleansing composition comprising:

(a) a cleansing surfactant comprising:

(iii) an acyl sarcosinate; and

(iv) an additional cleansing surfactant selected from the group consisting of an anionic surfactant other than the acyl sarcosinate, an amphoteric surfactant, a zwitterionic surfactant, and mixtures thereof;

wherein the acyl sarcosinate is contained at a level of from about 40% to about 90% by weight of the total cleansing surfactant, and at a level of from about 4% to about 25% by weight of the composition; and

(b) water.

### CROSS REFERENCE TO RELATED APPLICATIONS

This application claims the benefit of U.S. Provisional Application No. 60/365,455, filed on Mar. 19, 2002.

### TECHNICAL FIELD

The present invention relates to liquid personal cleansing compositions containing acyl sarcosinates at a high level.

### BACKGROUND

Skin is made up of several layers of cells which coat and protect the underlying tissue. The keratin and collagen fibrous proteins form the skeleton of its structure. The outermost of these layers, referred to as the stratum corneum.

Personal cleansing compositions can clean such skins. Some cleansing surfactants used in personal cleansing compositions penetrate the stratum corneum membrane, by delipidization destroy membrane integrity and loss of barrier and water retention functions. This interference with skin protective membranes can lead to a rough skin feel and eye irritation and may eventually permit the surfactant to trigger immune response creating irritation.

Thus, there is a need for cosmetic cleansing compositions which cleanse the skin gently, without disrupting structural lipids and/or drying the skin and without irritating the ocular mucosae or leaving skin taut after frequent use.

There is also a need for personal cleansing compositions which provide good lathering properties, and improved skin feel such as moisturized feel, softness, reduced sticky feel, and reduced slippery feel.

U.S. Pat. No. 4,772,424 discloses cleansing compositions comprising from about 15% to about 30% of anionic surfactants such as ethoxylated alkyl sulfates, from about 0.8% to about 15% of betain surfactants, and from about 0.5% to about 15% of sarcosinate surfactants. These compositions are not satisfactory in terms of reduced sticky feel and reduced slippery feel. For consumers who desire personal cleansing composition to provide clean feel and easily rinsed feel, sticky and slippery feel is not desirable.

Thus, there is still a need for personal cleansing compositions which provide good lathering properties, clean feel, easily rinsed feel, and mildness to skin, while providing improved skin feel such as moisturized feel and softness.

### SUMMARY

The present invention relates to a liquid personal cleansing composition comprising:

(a) a cleansing surfactant comprising:

(i) an acyl sarcosinate; and

(ii) an additional cleansing surfactant selected from the group consisting of an anionic surfactant other than the acyl sarcosinate, an amphoteric surfactant, a zwitterionic surfactant, and mixtures thereof;

wherein the acyl sarcosinate is contained at a level of from about 40% to about 90% by weight of the total cleansing surfactant, and at a level of from about 4% to about 25% by weight of the composition; and

(b) water.

The compositions of the present invention can provide good lathering properties, clean feel, easily rinsed feel, and mildness to skin, while providing improved skin feel such as moisturized feel and softness.

These and other features, aspects, and advantages of the present invention will become evident to those skilled in the art from a reading of the present invention.

### DETAILED DESCRIPTION

The following is a list of definitions for terms used herein.

“Comprising” means that other steps and other ingredients which do not affect the end result can be added. This term encompasses the terms “consisting of” and “consisting essentially of”.

All percentages are by weight of total composition unless specifically stated otherwise.

All cited references are incorporated herein by reference in their entireties. Citation of any reference is not an admission regarding any determination as to its availability as prior art to the claimed invention.

All ratios are weight ratios unless specifically stated otherwise.

Surfactant chain lengths are on a weight average chain length basis, unless otherwise specified.

As used herein the term “water-insoluble” in relation to oils is a material which is substantially insoluble in distilled water at room temperature without the addition of other adjuncts or ingredients such as those detailed herein.

The present invention, in its product and process aspects, is described in detail as follows.

Cleansing Surfactant

The liquid personal cleansing composition of the present invention comprises a cleansing surfactant. Cleansing surfactants of the present invention comprises acyl sarcosinates and additional cleansing surfactants. The additional cleansing surfactants are selected from the group consisting of anionic surfactants other than the acyl sarcosinate, amphoteric surfactants, zwitterionic surfactants, and mixtures thereof. Nonionic surfactants can be used as cleansing surfactants.

The acyl sarcosinate is contained at a level of from about 40% to about 90%, preferably from about 50% to about 90% by weight of the total cleansing surfactant. Additional cleansing surfactants can be contained at a level of from about 10% to about 60%, preferably from about 10% to about 50% by weight of the total cleansing surfactant.

The acyl sarcosinate is contained at a level of from about 4% to about 25% by weight of the composition, preferably from about 4% to about 20%, more preferably from about 6% to about 12%.

The total level of cleansing surfactants is preferably from about 8% to about 30%, more preferably from about 12% to about 25%, and still preferably from about 12% to about 20% by weight of the composition.

The total level of additional cleansing surfactant is preferably from about 2% to about 15%, more preferably from about 3% to about 12%, and still preferably from about 5% to about 10% by weight of the composition.

The additional cleansing surfactants preferably comprise a mixture of anionic surfactants and amphoteric surfactants. The weight ratio of anionic surfactants: amphoteric surfactants can be in the range of, preferably from about 1:10 to about 10:1, more preferably from about 1:5 to about 5:1, still more preferably from about 1:3 to about 3:1.

Acyl Sarcosinate

The liquid personal cleansing composition of the present invention comprises an acyl sarcosinate. Without intending to be limited by theory, it is believed that acyl sarcosinate surfactants can provide improved lathering properties and easily rinsed feel.

Acyl sarcosinates useful herein include, for example, those having a formula:

RCON(CH₃)CH₂COOM

wherein R is an alkyl or alkenyl having from 8 to 22 carbon atoms, preferably from 12 to 18 carbon atoms, more preferably from 12 to 14 carbon atoms; and M is sodium, potassium, ammonium, or triethanolamine.

Acyl sarcosinates preferably used herein include, for example, sodium lauroyl sarcosinate, sodium cocyl sarcosinate, sodium oleyl sarcosinate, and mixtures thereof. Commercially available preferred acyl sarcosinates include, for example, sodium lauroyl sarcosinate having tradename Hamposyl L30 available from Hampshire Chemicals, and sodium cocoyl sarcosinate having tradename Hamposyl C30 available from Hampshire Chemicals.

Anionic Surfactant

The composition of the present invention preferably comprises anionic surfactants other than the acyl sarcosinate. Without intending to be limited by theory, it is believed that the anionic surfactants can provide moisturized feel.

Anionic surfactants useful in the present invention include, for example, ethoxylated alkyl sulphates, alkyl ethoxy carboxylates, alkyl glyceryl ether sulphonates, alkyl ethoxysulphosuccinates, alpha sulphonated fatty acids, their salts and/or their esters, ethoxylated alkyl phosphate esters, ethoxylated alkyl glyceryl ether sulfonates, paraffin sulfonates and alkoxy amide sulfonates, alkyl sulphates, and mixtures thereof.

Among a variety of anionic surfactants, preferred are ethoxylated alkyl sulphates, such as ammonium laureth-3 sulfate, in view of moisturized feel.

Anionic surfactants is contained at a level of, by weight of the composition, preferably from about 1% to about 15%, more preferably from about 1% to about 10%, still more preferably from about 2% to about 8%.

Amphoteric Surfactant

The composition of the present invention preferably comprises amphoteric surfactants. Without intending to be limited by theory, it is believed that amphoteric surfactants can provide moisturized feel.

Amphoteric surfactants useful herein include, for example, ammonium derivatives of a formula:

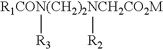

wherein R₁ is C₅-C₂₂ alkyl or alkenyl, R₂ is CH₂CH₂OH or CH₂CO₂M, M is H, alkali metal, alkaline earth metal, ammonium or alkanolammonium and R₃ is CH₂CH₂OH or H.

Such amphoteric surfactants include, for example, cocoamphocarboxypropionate, cocoamphocarboxy propionic acid, cocoamphoacetate, cocoamphodiacetate (otherwise referred to as cocoamphocarboxyglycinate), sodium lauroamphoacetate (otherwise referred to as sodium lauroamphocarboxyglycinate). Commercially available preferred amphoteric surfactants include, for example, those sold under the trade names of Ampholak 7TX (sodium carboxy methyl tallow polypropyl amine); Empigen CDL60, CDL60P, and CDR 60 (Albright & Wilson); Miranol H2M Conc., Miranol C2M Conc. N.P., Miranol C2M Conc. O.P., Miranol C2M SF, Miranol CM Special, Miranol Ultra L32 and C32 (Rhone-Poulenc); Alkateric 2CIB (Alkaril Chemicals); Amphoterge W-2 (Lonza, Inc.); Monateric CDX-38, Monateric CSH-32 (Mona Industries); Rewoteric AM-2C (Rewo Chemical Group); and Schercotic MS-2 (Scher Chemicals).

It will be understood that a number of commercially-available amphoteric surfactants of this type are manufactured and sold in the form of electroneutral complexes with, for example, hydroxide counterions or with anionic sulfate or sulfonate surfactants, especially those of the sulfated C₈-C₁₈ alcohol, C₈-C₁₈ ethoxylated alcohol or C₈-C₁₈ acyl glyceride types. Note also that the concentrations and weight ratios of the amphoteric surfactants are based herein on the uncomplexed forms of the surfactants, any anionic surfactant counterions being considered as part of the overall anionic surfactant component content.

Amphoteric surfactants are contained at a level of, by weight of the composition, preferably from about 1% to about 15%, more preferably from about 1% to about 10%, still more preferably from about 2% to about 8%.

Zwitterionic Surfactant

The compositions herein can also contain a zwitterionic surfactant. Without intending to be limited by theory, it is believed that zwitterionic surfactants can provide moisturized feel.

Zwitterionic surfactants can be contained at a level of, preferably from about 0.1% to about 15%, more preferably from about 0.1% to about 10%, and still preferably from about 0.1% to about 8% by weight of the composition.

Zwitterionic surfactants preferably used herein include, for example, betaine surfactants. Such betain surfactants useful herein include, for example, alkyl betaines of the formula R₅R₆R₇N⁺(CH₂)ₙCO₂M and amido betaines of the formula:

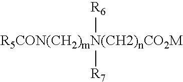

wherein R₅ is C₅-C₂₂ alkyl or alkenyl, R₆ and R₇ are independently C₁-C₃ alkyl, M is H, alkali metal, alkaline earth metal, ammonium or alkanolammonium, and n, m are each numbers from 1 to 4. Preferred betaines include, for example, cocoamidopropyldimethylcarboxymethyl betaine, commercially available from TH Goldschmidt under the tradename Tego betaine, and laurylamidopropyldimethylcarboxymethyl betaine, commercially available from Albright and Wilson under the tradename Empigen BR and from TH Goldschmidt under the tradename Tegobetaine L10S.

Nonionic Surfactant

The compositions according to the present invention may include a nonionic surfactant. Nonionic surfactants can be contained at a level of, preferably from about 0.1% to about 15%, more preferably from about 0.1% to about 10%, and still preferably from about 0.1% to about 8% by weight of the composition.

Nonionic surfactants useful herein include, for example, sucrose polyester surfactants, C₁₀-C₁₈ alkyl polyglycosides and polyhydroxy fatty acid amide surfactants.

Among them, preferred nonionic surfactants are polyhydroxy fatty acid amide surfactants. The more preferred polyhydroxy fatty acid amide has the formula R₈(CO)N(CH₃)CH₂(CHOH)₄CH₂OH wherein R₈ is a C₆-C₁₉ straight chain alkyl or alkenyl group. In compounds of the above formula, R₈—CO—N< can be, for example, cocoamide, stearamide, oleamide, lauramide, myristamide, capricamide, caprylicamide, palmitamide, tallowamide, etc. Such polyhydroxy fatty acid amide surfactants useful herein include, for example, primary amines such as cocamine (available as Adagen 160D (™) from Witco) and alkanolamides such as cocamide MEA (available as Empilan CME (™) from Albright and Wilson), PEG-3 cocamide, cocamide DEA (available as Empilan CDE (™) from Albright and Wilson), lauramide MEA (available as Empilan LME (™) from Albright and Wilson), lauramide MIPA, lauramide DEA, and mixtures thereof.

Water

The composition of the present invention comprises water. Water can be present at a level preferably of from about 20% to about 99.89%, more preferably from about 40% to about 90%, still more preferably at least about 75% by weight of the compositions.

Suspending Agent

The compositions of the present invention can contain one or more suspending agents. The suspending agents can serve as a thickener, and the suspending agents can serve to assist in suspending the water-insoluble oil and may give pearlescence to the product, when the oil is contained in the composition.

Suitable suspending agents for use herein include any of several long chain acyl derivative materials or mixtures of such materials. Included are ethylene glycol esters of fatty acids having from about 16 to about 22 carbon atoms. Preferred are the ethylene glycol stearates, both mono and distearate, but particularly the distearate containing less than about 7% of the mono stearate. Other suspending agents found useful are alkanol amides of fatty acids, having from about 16 to 22 carbon atoms, preferably from about 16 to 18 carbon atoms. Preferred alkanol amides are stearic monoethanolamide, stearic diethanolamide, stearic monoisopropanolamide and stearic monoethanolamide stearate.

Other suitable suspending agents are alkyl (C₁₆-C₂₂) dimethyl amine oxides such as stearyl dimethyl amino oxide and trihydroxystearin commercially available under the tradename Thixcin (RTM) from Rheox.

Mixtures of suspending agents are also suitable for use in the compositions of this invention.

The suspending agent can be contained at a level of, preferably from about 0.1% to about 5%, more preferably from about 0.2% to about 3%, still more preferably from about 0.3 to about 1.0% by weight of the composition.

Opacifier or Pearlescing Agent

The compositions according to the present invention can contain an opacifier or pearlescing agent. Such materials can be contained at a level of, preferably from about 0.01% to about 5%, more preferably from about 0.05% to about 1.0% by weight of the composition.

Opacifiers/pearlescers suitable for inclusion in the compositions of the present invention include, for example: titanium dioxide (TiO₂); long chain (C₁₆-C₂₂) acyl derivatives such as glycol or polyethylene glycol esters of fatty acid having from about 16 to about 22 carbon atoms and up to 7 ethyleneoxy units; alkanolamides of fatty acids, having from about 16 to about 22 carbon atoms, preferably about 16 to 18 carbon atoms such as stearic monoethanolamide, stearic diethanolamide, stearic monoisopropanolamide and stearic monoethanolamide and alkyl (C₁₆- C₂₂) dimethyl amine oxides such as stearyl dimethyl amine oxide; a particulate dispersion of polystyrene such as styrene acrylate copolymer having a particle size of from about 0.05 microns to about 0.45 microns.

Hydrotrope

The compositions according to the present invention may include a hydrotrope. Suitable for use herein as hydrotropes are those well known in the art, including sodium xylene sulphonate, ammonium xylene sulphonate, sodium cumene sulphonate, short chain alkyl sulphate and mixtures thereof. Hydrotrope may be present in the compositions according to the invention at a level of from about 0.01% to about 5%, preferably from about 0.1% to about 4%, more preferably from about 0.5% to about 3% by weight of the composition. Hydrotrope, as defined herein, means a material which can modify its viscosity and rheological profile when added to a surfactant mixture.

Water-Insoluble Polyalphaolefin Oil

The composition of the present invention can contain a water-insoluble polyalphaolefin oil.

Such water-insoluble polyalphaolefin oils useful herein include, for example, highly branched polyalphaolefins having the following formula (a):

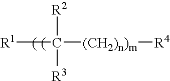

wherein R¹ is H or C₁-C₂₀ alkyl, R⁴ is C₁-C₂₀ alkyl, R² is H or C₁-C₂₀, and R³ is C₃-C_{20,} preferably C₅-C₂₀, n is an integer from 0 to 3 and m is an integer of from 1 to 1000 and having a number average molecular weight of from about 1000 to about 25,000, preferably from about 2500 to about 6000, more preferably from about 2500 to about 4000. Preferably the polyalphaolefins of type (a) have a viscosity of from about 300 cSt to about 50,000 cSt, preferably from about 1000 cSt to about 12,000 cSt, more preferably from about 1000 cSt to about 4000 cSt at 40° C. using the ASTM D-445 method for measuring viscosity. The oils of type (a) may also have a degree of unsaturation, but are preferably saturated.

Suitable polyalphaolefins of type (a) as described above can be derived from 1-alkene monomers having from about 4 to about 20 carbon atoms, preferably from about 6 to about 12 carbon atoms, especially from about 8 to about 12 carbon atoms. The polyalphaolefins useful herein are preferably hydrogenated polyalphaolefin polymers.

Non-limiting examples of 1-alkene monomers for use in preparing the polyalphaolefin polymers herein include 1-hexene, 1-octene, 1-decene, 1-dodecene, 1-tetradecene, branched chain isomers such as 4-methyl-1-pentene, and combinations thereof. Also suitable for preparing the polyolefin liquids are 1-hexene to 1-hexadecenes and combinations thereof, more preferably 1-octene to 1-dodecene or combinations thereof.

Examples of such oils include polydecene oils such as those commercially available from Mobil Chemical Company, P.O. Box 3140, Edison, N.J. 08818, USA, under the tradename Puresyn 40, Puresyn 100, and_Puresyn 101LT. Particularly preferred are those having a number average molecular weight of from about 2500 to about 4000 and a viscosity of from about 100 cSt to about 2000 cSt (ASTM D-445 at 45° C.) such as that commercially available from Mobil under the tradename Puresyn 100 and Puresyn 101LT.

In preferred embodiments the number average particle diameter for the water-insoluble oil used herein lies in the range of from about 1 micron to about 500 microns, preferably from about 5 to about 200 microns, more preferably from about 5 to 50 microns, especially from about 5 to about 20 microns.

The water-insoluble polyalphaolefin oil is contained at a level of, preferably from about 0.01% to about 5%, more preferably from about 0.1% to about 3%, by weight of the composition.

Polymeric Cationic Conditioning Agent

The compositions according to the present invention may include a polymeric cationic conditioning agent. The polymeric skin conditioning agent can be present at a level of, preferably from about 0.01% to about 5%, more preferably from about 0.01% to about 3% and still more preferably from about 0.01% to about 2% by weight of the composition.

Suitable polymers are high molecular weight materials (mass-average molecular weight determined, for instance, by light scattering, being generally from about 2,000 to about 5,000,000, preferably from about 5,000 to about 3,000,000 more preferably from 100,000 to about 1,000,000).

Representative classes of polymers include, for example, cationic guar gums, cationic polysaccharides; cationic homopolymers and copolymers derived from acrylic and/or methacrylic acid; cationic cellulose resins, quaternized hydroxy ethyl cellulose ethers; cationic copolymers of dimethyldiallylammonium chloride and acrylamide and/or acrylic acid; cationic homopolymers of dimethyldiallylammonium chloride; copolymers of dimethyl aminoethylmethacrylate and acrylamide, copolymers of dimethyldiallylammonium chloride and acrylamide, acrylic acid/dimethyldiallylammonium chloride/acrylamide copolymers, quaternised vinyl pyrrolidone acrylate or methacrylate copolymers of amino alcohol, quaternized copolymers of vinyl pyrrolidone and dimethylaminoethylmethacrylamide, vinyl pyrollidone/vinyl imidazolium methochloride copolymers and polyalkylene and ethoxypolyalkylene imines; quaternized silicones, terpolymers of acrylic acid, methacrylamidopropyl trimethyl ammonium chloride and methyl acrylate, and mixtures thereof.

Polymeric Thickening Component

The present compositions may include an auxiliary nonionic or anionic polymeric thickening component, especially a water-soluble polymeric materials, having a molecular weight greater than about 20,000. By “water-soluble polymer” is meant that the material will form a substantially clear solution in water at a 1% concentration at 25° C. and the material will increase the viscosity of the water. Examples of water-soluble polymers which may be used as an additional thickening component in the present compositions, are hydroxyethylcellulose, hydroxypropyl cellulose, hydroxypropyl methylcellulose, polyethylene glycol, polyacrylamide, polyacrylic acid, polyvinyl alcohol (examples include PVA 217 from Kurary Chemical Co., Japan), polyvinyl pyrrolidone K-120, dextrans, for example Dextran purified crude Grade 2P, available from D&O Chemicals, carboxymethyl cellulose, plant exudates such as acacia, ghatti, and tragacanth, seaweed extracts such as sodium alginate, propylene glycol alginate and sodium carrageenan. Preferred as the additional thickeners for the present compositions are natural polysaccharide materials. Examples of such materials are guar gum, locust bean gum, and xanthan gum. Also suitable herein preferred is hydroxyethyl cellulose having a molecular weight of about 700,000.

The polymeric thickening component can be present at a level of, preferably from about 0.01% to about 5%, more preferably from about 0.01% to about 3% and still more preferably from about 0.01% to about 2% by weight of the composition.

Other Oily Component

In addition to the water-insoluble polyalphaolefin oil described above, the compositions of the invention may also include cosmetic oils, waxes, or mixtures thereof at a level up to about 10%, preferably up to about 3% by weight of the composition, wherein the oil or wax is insoluble in the sense of being insoluble in the product matrix at a temperature of 25° C.

Suitable insoluble cosmetic oils and waxes for use herein can be selected from water-insoluble silicones inclusive of non-volatile polyalkyl and polyaryl siloxane gums and fluids, volatile cyclic polydimethylsiloxanes, polyalkoxylated silicones, amino and quaternary ammonium modified silicones, rigid cross-linked and reinforced silicones and mixtures thereof, C₁-C₂₄ esters of C₈-C₃₀ fatty acids such as isopropyl myristate, myristyl myristate and cetyl ricinoleate, C₈-C₃₀ esters of benzoic acid, beeswax, saturated and unsaturated fatty alcohols such as behenyl alcohol, hydrocarbons such as mineral oils, petrolatum, squalane and squalene, fatty sorbitan esters (see U.S. Pat. No. 3,988,255, Seiden, issued Oct. 26, 1976), lanolin and oil-like lanolin derivatives, animal and vegetable triglycerides such as almond oil, peanut oil, wheat germ oil, rice bran oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soyabean oil, avocado oil, safflower oil, coconut oil, hazelnut oil, olive oil, grapeseed oil, and sunflower seed oil, and C₁-C₂₄ esters of dimer and trimer acids such as diisopropyl dimerate, diisostearylmalate, diisostearyldimerate and triisostearyltrimerate.

Moisturizer

The cleansing compositions may include other skin moisturizers which are soluble in the cleansing composition matrix. The preferred level of such moisturizers is from about 0.01% to about 20% by weight of the composition. In preferred embodiments, the moisturizer is selected from essential amino acid compounds found naturally occurring in the stratum corneum of the skin and water-soluble nonpolyol nonocclusives and mixtures thereof.

Some examples of more preferred nonocclusive moisturizers are squalane, sodium pyrrolidone carboxylic acid, D-panthenol, lactic acid, L-proline, guanidine, pyrrolidone, hydrolyzed protein and other collagen-derived proteins, aloe extract including aloe vera gel, acetamide MEA and lactamide MEA and mixtures thereof.

Additional Components

A number of additional materials can be added to the cleansing compositions each at a level of from about 0.01% to about 2% by weight of the composition. Such materials include proteins and polypeptides and derivatives thereof; water-soluble or solubilizable preservatives such as Kathon CG available from Rohm&Haas, DMDM Hydantoin, Germall 115, methyl paraben, ethyl paraben, propyl paraben, butyl paraben, EDTA, Euxyl (RTM) K400, natural preservatives such as benzyl alcohol, potassium sorbate and bisabalol; sodium benzoate and 2-phenoxyethanol; other moisturizing agents such as hyaluronic acid, chitin, and starch-grafted sodium polyacrylates such as Sanwet (RTM) IM-1000, IM-1500 and IM-2500 available from Celanese Superabsorbent Materials, Portsmith, Va. USA and described in U.S. Pat. No. 4,076,663; solvents; suitable anti-bacterial agents such as Oxeco (phenoxy isopropanol), Trichlorocarbanilide (TCC) and Triclosan and; low temperature phase modifiers such as ammonium ion sources (e.g. NH₄ Cl); viscosity control agents such as magnesium sulfate and other electrolytes; colouring agents; TiO₂-coated mica; perfumes and perfume solubilizers; and zeolites such as Valfour BV400 and derivatives thereof; Ca²⁺/Mg²⁺ sequestrants such as polycarboxylates, amino polycarboxylates, polyphosphonates, amino polyphosphonates, EDTA etc; water softening agents such as sodium citrate; insoluble particulates such as zinc stearate and fumed silica; infrared screening and absorbing agents; film forming polymers such as polyvinyl pyrrolidone; guai azulene; saccharomycopsis ferment filtrate; coix lacryma-jobi (job's tears) seed extract; salicylic acid; and others.

Composition

In preferred embodiments, the compositions are substantially free of soap, i.e. they contain less than about 5%, preferably less than about 1%, preferably 0%, by weight of soap. Soaps herein include those such as ammonium, magnesium, potassium, triethanolamine and sodium salts of lauric acid, myristic acid and palmitic acid, acyl aspartates, alkoxy cocamide carboxylates, (ethoxylated) alkanolamide sulphosuccinates, ethoxylated alkyl citrate sulphosuccinates, acyl ethylene diamine triacetates, acylhydroxyethyl isethionates, acyl amide alkoxy sulfates, linear alkyl benzene sulfonates, paraffin sulfonates, alpha olefin sulfonates, alkyl alkyoxy sulfates, and mixtures thereof. Alkyl and/or acyl chain lengths for these surfactants are C₆-C₂₂.

The pH of the compositions is preferably from about 3 to about 10, more preferably from about 5 to about 9, especially from about 5 to about 8 and most preferably from about 5 to 7.

The viscosity of the final composition (Brookfield DV II, with Cone CP41 or CP52, 25° C., neat) is preferably at least about 500 cps, more preferably from about 1,000 to about 50,000 cps, especially from about 1,000 to about 30,000 cps, more especially from about 1,000 to about 10,000 cps.

The compositions of the present invention can be used for a variety of skin and hair care applications such as shower gels, body washes, hair shampoos, and the like. The compositions of the present invention may be applied with the hand or preferably with a personal cleansing implement such as a puff.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

All percentages herein are based upon the total weight of the compositions, and all such weight percentages as they pertain to listed ingredients are based on the active level and, therefore, do not include carriers or by-products that may be included in commercially available materials.

Compositions

| | Component | EX. 1 | EX. 2 | Ex. 3 | Ex. 4 | Ex. 5 |
|---|---|---|---|---|---|---|
| 1 | Sodium Lauroyl | 7.5 | 6.5 | 6.5 | 11.25 | 7.5 |
| | Sarcosinate *1 | | | | | |
| 2 | Ammonium Laur- | 3.75 | 3.25 | 6.5 | — | 3.75 |
| | eth-3 Sulphate *2 | | | | | |
| 3 | Lauroamphoacetate | 3.75 | 3.25 | — | 3.75 | 2.0 |
| | *3 | | | | | |
| 4 | Cocoamidopropyldi- | — | — | — | — | 1.75 |
| | methyl carboxy- | | | | | |
| | methyl Betaine *4 | | | | | |
| 5 | Trihydroxy- | 0.4 | 0.7 | 0.7 | 0.5 | 0.4 |
| | stearin *5 | | | | | |
| 6 | Lauryl Alcohol | — | 0.5 | 0.5 | 0.25 | 0.2 |
| 7 | Titanium Dioxide | 0.1 | 0.1 | 0.1 | 0.1 | 0.1 |
| | *6 | | | | | |
| 8 | Polydecene *7 | — | 0.1 | — | — | — |
| 9 | Guar Hydroxy- | — | — | — | — | 0.7 |
| | propyl Trimonium | | | | | |
| | Chloride *8 | | | | | |
| 10 | Aloe Extract *9 | 0.0005 | 0.0005 | 0.0005 | 0.0005 | 0.0005 |
| 11 | Trichlorocarbanilide | 0.15 | 0.15 | 0.15 | 0.15 | 0.15 |
| | *10 | | | | | |
| 12 | Niacinamide *11 | 0.05 | 0.05 | 0.05 | 0.05 | 0.05 |
| 13 | Penthenol *12 | 0.05 | 0.05 | 0.05 | 0.05 | 0.05 |
| 14 | Guai-azulene *13 | 0.05 | 0.05 | 0.05 | 0.05 | 0.05 |
| 15 | Saccharomycopsis | 0.05 | 0.05 | 0.05 | 0.05 | 0.05 |
| | Ferment Filtrate *14 | | | | | |
| 16 | Coix Lacryma-jobi | 0.05 | 0.05 | 0.05 | 0.05 | 0.05 |
| | (Job's Tears) Seed | | | | | |
| | Extract *15 | | | | | |
| 17 | Perfume | 0.8 | 0.8 | 0.8 | 0.8 | 0.8 |
| 18 | Disodium EDTA | 0.1 | 0.1 | 0.1 | 0.1 | 0.1 |
| 19 | Sodium Benzoate | 0.25 | 0.25 | 0.25 | 0.25 | 0.25 |
| 20 | Kathon CG *16 | 0.033 | 0.033 | 0.033 | 0.033 | 0.033 |
| 21 | NaCl | 3.6 | 3.6 | 3.6 | 3.6 | 3.6 |
| 22 | Citric acid | 0.9 | 0.9 | 0.6 | 0.9 | 0.9 |

| | |
|---|---|
| Water | q.s. to 100% |

Definitions of Components
*1 Sodium Lauroyl Sarcosinate: Hamposyl L30 available from Hampshire Chemicals
*2 Ammonium laureth-3 sulphate: Empicol EAC/TP available from Albright & Wilson
*3 Lauroamphoacetate: Empigen CDL60P available from Albright & Wilson
*4 Cocoamidopropyldimethylcarboxymethyl betaine: Tego betaine available from TH Goldschmidt
*5 Trihydroxystearin: Thixcin available from Rheox
*6 Titanium Dioxide: 3328 Titanium Dioxide available from Whittaker Clark & Daniels
*7 Polydecene: Puresyn 100 available from Mobil Chemical Co.
*8 Guar Hydroxypropyl Trimonium Chloride: N-Hance Cationic Guar available from Aqualon
*9 Aloe extract: Aloe Vera 1:1 available from Mexi Aloe
*10 Trichlorocarbanilide: Nipaguard TCC available from Nipaguard Laboratories
*11 Niacinamide: Niacinamide available from Roche
*12 Panthenol: DL-Panthenol available from Roche
*13 Guai;azulene: available from Konan Kako
*14 Saccharomycopsis Ferment Filtrate: SK-II Pitera available from Kashiwayama

*15 Coix Lacryma-jobi (Job's Tears) Seed Extract: Coix Seed Extract available from Koei Kogyo

*16 Kathon CG: Available from Rohm&Haas

Method of Preparation

The compositions of Examples 1-5 are prepared as follows: component numbers 1 through 5, 18, 19, 22 and some part of water are mixed and heated to dissolve at 91° C. and then cooled to 70° C. or below. Then, component numbers 6 through 17, 20, and remaining water are added into the mixture. Among these components, water soluble components such as Niacinamide and Panthenol are preferably added into the mixture as a water solution, and guar hydroxypropyl trimonium chloride are preferably added into the mixture as a water slurry. Finally, component number 21 is added into the mixture. The compositions of Examples 1-5 provide a liquid personal cleansing composition.

The embodiments disclosed herein have many advantages. For example, the liquid personal cleansing compositions of Examples 1 through 5 as shown above provide good lathering properties, clean feel, easily rinsed feel, and mildness to skin, while providing improved skin feel such as moisturized feel and softness.

It is understood that the examples and embodiments described herein are for illustrative purposes only and that various modifications or changes in light thereof will be suggested to one skilled in the art without departing from its spirit and scope.

What is claimed is:

1. A liquid personal cleansing composition comprising:
(a) a cleansing surfactant comprising:
(i) an acyl sarcosinate; and
(ii) an additional cleansing surfactant selected from the group consisting of an anionic surfactant other than the acyl sarcosinate, an amphoteric surfactant, a zwitterionic surfactant, and mixtures thereof;
wherein the acyl sarcosinate is contained at a level of from about 40% to about 90% by weight of the total cleansing surfactant, and at a level of from about 4% to about 25% by weight of the composition; and
(b) water.

2. The liquid personal cleansing composition according to claim 1, wherein the acyl sarcosinate is contained at a level of from about 50% to about 90% by weight of the total cleansing surfactant.

3. The liquid personal cleansing composition according to claim 1, wherein the additional cleansing surfactant comprises the anionic surfactant and the amphoteric surfactant.

4. The liquid personal cleansing composition according to claim 1, wherein the anionic surfactants other than the acyl sarcosinate is an ethoxylated alkyl sulphate.

5. The liquid personal cleansing composition according to claim 1, wherein the anionic surfactant other than the acyl sarcosinate is contained at a level by weight of from about 1% to about 15% by weight of the composition.

6. The personal cleansing composition according to claim 1, wherein the amphoteric surfactant is selected from ammonium derivatives of formula [V]: 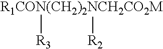
wherein R₁ is C₅-C₂₂ alkyl or alkenyl, R₂ is CH₂CH₂OH or CH₂CO₂M, M is H, alkali metal, alkaline earth metal, ammonium or alkanolammonium and R₃ is CH₂CH₂OH or H.

7. The liquid personal cleansing composition according to claim 1, wherein the composition is substantially free of soap.

8. The liquid personal cleansing composition according to claim 1 further comprising from about 0.01% to about 5% by weight of a water-insoluble polyalphaolefin oil.
